(19) United States
(12) Patent Application Publication
(10) Pub. No.: US 2025/0099418 A1
(43) Pub. Date: Mar. 27, 2025

(54) COMPOSITION HAVING EFFECTS OF PROTECTING EYES AND IMPROVING EYESIGHT AND USE THEREOF IN FIELD OF PREPARING FOOD OR MEDICINE

(71) Applicant: Renhe Global (Shanghai) Grand Health Research Institute Co., Ltd, Shanghai (CN)

(72) Inventors: Shengquan Huang, Shanghai (CN); Xiaomi Wang, Shanghai (CN); Yunxiao Shao, Shanghai (CN); Xiaolan Wang, Shanghai (CN)

(21) Appl. No.: 18/590,939

(22) Filed: Feb. 29, 2024

(30) Foreign Application Priority Data
Sep. 22, 2023 (CN) 202311228471.3

(51) Int. Cl. A61K 31/235; A61K 9/50; A61K 31/185; A61K 31/375; A61K 31/728; A61K 36/287; A61K 36/736; A61K 47/36; A61P 27/02

(52) U.S. Cl. CPC A61K 31/235; A61K 9/50; A61K 31/185; A61K 31/375; A61K 31/728; A61K 36/287; A61K 36/736; A61K 47/36; A61P 27/02

(57) ABSTRACT

The present application discloses a composition with effects of protecting eyes and improving eyesight and use thereof in the field of preparing food or medicines, and relates to the field of functional food. The composition includes lutein ester microcapsule powder, wild cherry berry juice concentrate, black currant juice concentrate, sodium hyaluronate, vitamin C, taurine and Hang white chrysanthemum. The composition of the present application eliminates free radicals by antioxidant components, has effects of improving and repairing retina, protecting ocular microvessels, improving fundus microcirculation, and increasing ciliary muscle blood flow, and is supplemented with sodium hyaluronate and taurine to maintain corneal shape. The functional components are formulated to generate a synergistic effect, so that the composition can eliminate the symptom of asthenopia, and can be applied to medical health products or food to relieve asthenopia.

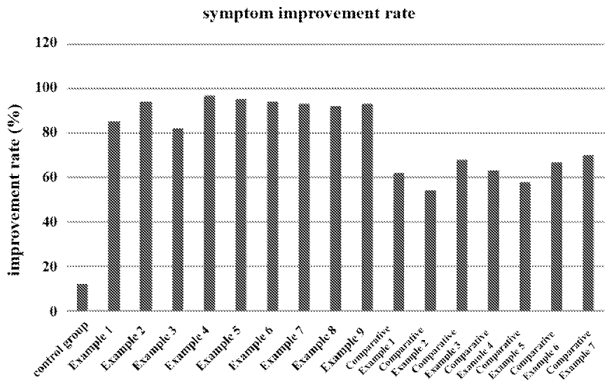

### CROSS-REFERENCE TO RELATED APPLICATION

The present application claims priority from Chinese Patent Application No. 202311228471.3 filed on Sep. 22, 2023, the contents of which are incorporated herein by reference in their entirety.

### TECHNICAL FIELD

The application relates to the field of functional food, in particular to a composition having effects of protecting eyes and improving eyesight and use thereof in the field of preparing food or medicines.

### BACKGROUND

In recent years, with the increasing development of electronic science and technology, people contact the screen of computers, mobile phones and other electronic products more and more frequently. Due to staring at the screen for a long time, the number of blinks decreases, resulting in a corresponding reduction in tear secretion. At the same time, the flashing screen strongly stimulates the eyes, and it is easy to cause asthenopia if the eyes do not take a rest in time. Asthenopia causes dry eyes, eye astringence, eye distension and blurred vision, which directly affects people's work and life, and even seriously causes diminution of vision, formation of myopia, amblyopia, and asthenopia or occurrence of eye soreness, orbital pain and other diseases.

At present, there are many ways to relieve asthenopia, mainly including increasing the rest time of eyes, taking health products for relieving asthenopia and using eye drops. However, nowadays life is stressful, and people are always too busy and cannot let the eyes get enough rest. If the symptoms are not particularly serious, for example there are no symptoms such as red eyes or inflammation, people are generally reluctant to use eye drops. At present, the health care products for relieving asthenopia are mainly based on raw materials such as blueberry and lutein, the product homogeneity phenomenon is serious, and the improvement degree of people with different physique is inconsistent. People have no more other options.

### SUMMARY

The present application provides a composition having effects of protecting eyes and improving eyesight, and use thereof in the field of preparing food or medicines, which contains anthocyanin as an effect component and effectively relieves the problem of asthenopia and dry eyes.

In order to solve the above technical problems, the first object of the present application is to provide a composition having effects of protecting eyes and improving eyesight, comprising the following components by weight:
lutein ester microcapsule powder: 0.1-1.5 parts;
wild cherry berry (Aornia mealnocarpa Elliot) juice concentrate: 2-15 parts;
black currant (Ribes nigrum L.) juice concentrate: 2-15 parts;
sodium hyaluronate: 0.1-2 parts;
vitamin C: 0.02-1 part;
taurine: 0.02-1.5 parts; and
Hang white chrysanthemum (Chrysanthemum morifolium Ramat.): 1-10 parts.

The present composition is based on specific ingredients, such as lutein ester microcapsule powder, wild cherry berry juice concentrate, black currant juice concentrate, sodium hyaluronate, vitamin C, taurine, and Hang white chrysanthemum. Lutein has physiological effects such as antioxidant, and can prevent visual loss caused by lesions in the macular area of the fundus. Wild cherry berry fruit contains vitamins, folate, quinic acid, polyphenolic acids, tannin, catechins, hesperidin and anthocyanins, etc., as well as rich flavonoids, and phenolic compounds like anthocyanins, so that wild cherry berry has strong antioxidant activity and free radical scavenging effect. Black currant fruit is rich in anthocyanins, polyphenols, vitamin C and other nutrients, and has effects of protecting eyesight and anti-oxidation. Sodium hyaluronate plays an important role in maintaining corneal shape. Taurine has an effect of maintaining and regulating visual function. The flavonoids, polyphenols and terpenoids contained in Hang white chrysanthemum have anti-free radical and antioxidant activities, thus protecting the eyes and relieving asthenopia. Through the reasonable proportion of a variety of raw materials having effects of protecting eyes and improving eyesight, free radicals can be removed to improve and repair the retina, while sodium hyaluronate and taurine can be used to maintain the shape of the cornea. The synergistic effect of the components can relieve asthenopia through multiple channels, effectively improving eye vision and relieving asthenopia.

In order to solve the above technical problems, the second object of the present application is to provide use of the composition having effects of protecting eyes and improving eyesight in the field of preparing food or medicines.

In order to solve the above technical problems, the third object of the present application is to provide a liquid preparation having effects of protecting eyes and improving eyesight, comprising the following components by weight:
lutein ester microcapsule powder: 0.1-1.5 parts;
wild cherry berry (Aornia mealnocarpa Elliot) juice concentrate: 2-15 parts;
black currant (Ribes nigrum L.) juice concentrate: 2-15 parts;
sodium hyaluronate: 0.1-2 parts;
vitamin C: 0.02-1 part;
taurine: 0.02-1.5 parts;
Hang white chrysanthemum (Chrysanthemum morifolium Ramat.): 1-10 parts;
a thickening agent: 0.4-3 parts; and
a solvent: 50-90 parts.

As a preferred embodiment, the thickening agent is pectin and/or xanthan gum, and the solvent is water.

As a preferred embodiment, the liquid preparation further comprises a sweetening agent in 0.01 to 9 parts by weight.

As a preferred embodiment, the sweetening agent is erythritol and/or sucralose.

As a preferred embodiment, the liquid preparation further comprises an acidity regulator of 0.02 to 0.8 parts by weight.

As a preferred embodiment, the acidity regulator is citric acid and/or sodium citrate.

As a preferred embodiment, the liquid preparation further comprises a flavoring agent of 0.1-0.3 parts by weight.

In order to solve the above technical problems, the fourth object of the present application is to provide a preparation method of the liquid preparation having effects of protecting eyes and improving eyesight, comprising the following steps:
S1: heating one-third of a solvent to 50° C.-70° C., mixing sodium hyaluronate and a sweetening agent evenly and adding to the solvent under stirring, heating a result mixture under stirring until the result mixture is transparent, and conducting cooling to obtain a mixture A;
S2: adding lutein ester microcapsule powder and Hang white chrysanthemum to another one-third of the solvent, and stirring evenly to obtain a mixture B;
S3: adding vitamin C, taurine and the acidity regulator into another one-third of the solvent, and stirring evenly to obtain a mixture C;
S4: adding the mixture B, wild cherry berry juice concentrate, black currant juice concentrate and the mixture C to the mixture A, and adding a flavoring agent and stirring evenly to homogenize to obtain the liquid preparation.

Compared with the prior art, the embodiment of the present application has the following beneficial effects:

(1) The composition provided by the present application, through the coordination of various functional components, has the effect of pacifying liver and clearing heat, and enriching kidney and improving eyesight, and can effectively reduce damage of free radicals to eyeball tissues, protect eye microvessels, improve fundus microcirculation, and increase ciliary muscle blood flow, thereby eliminating symptoms of asthenopia and improving vision. Therefore, the present composition can be applied to medical health products or food for relieving asthenopia to improve the body's health.

(2) The lutein ester microcapsule powder, wild cherry berry, blackcurant, vitamin C, Hang white chrysanthemum, etc. provided by the present application have antioxidant effects, can improve and repair retina by removing free radicals, and are supplemented with sodium hyaluronate and taurine to maintain corneal shape, and the obtained composition has the characteristics of food safety, no toxic side effects, and stable and controllable quality.

### BRIEF DESCRIPTION OF THE DRAWINGS

The sole FIGURE is a statistical chart showing the symptom improvement rates of liquid preparations in examples and comparative examples of the present application in relieving asthenopia.

### DETAILED DESCRIPTION

The technical solutions in the embodiment of the present application will be clearly and completely described below following in combination with the drawings attached to the embodiment of the present application. Obviously, the described embodiments are only a part of the embodiment of the present application, but not all of embodiments. Based on the embodiments of the present application, all other embodiments obtained by ordinary technicians in the field without creative labor fall within the scope of protection of the present application.

It is understood that the liquid preparations of the examples of the present application can also be prepared into various suitable dosage forms by adding different acceptable excipients for food or medicines, including but not limited to the following dosage forms: a tablet, a capsule, an oral liquid, an oral granule, an oral dispersion, or the like. The acceptable excipients for food or medicines include but are not limited to a diluent, a wetting agent, an adhesive, a disintegrant, a lubricant, a color and flavor modulator, a solvent, a solubilizer, a co-solvent, an emulsifier, an antioxidant, a metal complexing agent, a preservative, a pH regulator, etc. Further, the diluent includes starch, sucrose, celluloses, inorganic salts, etc.; the wetting agent includes water, ethanol, etc.; the adhesive includes starch pulp, dextrin, sugar, cellulose derivatives, gelatin, povidone, polyethylene glycol, etc.; the disintegrant includes starch, sodium carboxymethyl starch, low substituted hydroxypropyl cellulose, croscarmellose sodium, surfactants, etc.; the lubricant includes talc powder, calcium stearate, magnesium stearate, magnesium dodecyl sulfate, polyethylene glycol, etc.; the color and flavor modulator includes a pigment, a sweetening agent, flavor, a mucilage etc.; the solvent includes water, glycerin, ethanol, etc.; the solubilizer includes tweens, meizers, sulfates, sulfonates, etc.; the co-solvent includes organic acids (such as citric acid) and salts thereof, inorganic salts, polyethylene glycol, etc.; the emulsifier includes spans, glycerol fatty acid esters, gum Arabic, gelatin, agar, sodium alginate, etc.; the antioxidant includes sulfites, ascorbic acid, and gallic acid and salts thereof; the metal complexing agent includes disodium ethylenediamine tetraacetate, polycarboxylic acid compounds, etc.; the preservative includes nipagins, a quaternary ammonium compound, chlorhexidine acetate, etc.; and the pH regulator includes hydrochloric acid, tartaric acid, acetic acid, sodium hydroxide, sodium bicarbonate, ethylenediamine, meglumine, phosphate, citrate, etc.

The sources of raw materials in Examples or Comparative Examples of the present application are shown in Table 1. The used materials and reagents are commercially available unless otherwise specified.

TABLE 1
Sources and models of raw materials in Examples or
Comparative Examples of the present application

| Name of raw material | Manufacturer |
|---|---|
| Lutein ester microcapsule | Dalian Yinuo Biological Co., LTD |
| powder | |
| Wild cherry berry juice | Guangzhou Kanglai Food Additives Co., |
| concentrate | LTD |
| Blueberry juice | Ningxia Vanilla Biotechnology Co., LTD |
| concentrate | |
| Black currant juice | Bikas Trading (Shanghai) Co., LTD |
| concentrate | |
| Grape juice concentrate | Tianjin Yuchang Technology Development |
| | Co., LTD |
| Sodium hyaluronate | Shandong focus Freda Biological Co., LTD |
| Vitamin C | Hailongjiang Xinhecheng Biotechnology |
| | Co., LTD |
| Taurine | Qianjiang Yongan Pharmaceutical Co., LTD |
| Hang white chrysanthemum | Guangdong Qingyunshan Pharmaceutical |
| | Co., LTD |

### Examples 1-9

A liquid preparation having effects of protecting eyes and improving eyesight, with the components and contents shown in Table 2 below, comprised lutein ester microcapsule powder, wild cherry berry juice concentrate, black currant juice concentrate, sodium hyaluronate, vitamin C, taurine, Hang white chrysanthemum, a thickening agent, a sweetening agent, an acidity regulator, a flavoring agent and water, wherein the thickening agent was pectin or xanthan gum, the sweetening agent was erythritol and sucralose, the acidity regulator was citric acid and sodium citrate, and the flavoring agent was raspberry essence.

The preparation method of the liquid preparation having effects of protecting eyes and improving eyesight comprised the following steps:
S1: one-third of water was heated to 60° C., then sodium hyaluronate and a sweetening agent were mixed evenly and added to the one-third of water under stirring, and a resulting mixture was stirred and heated until the resulting mixture is transparent, and then cooled to obtain a mixture A;
S2: lutein ester microcapsule powder and Hang white chrysanthemum were added to another one-third of the water, and stirred evenly to obtain a mixture B;
S3: vitamin C, taurine and an acidity regulator were added into another one-third of the water, and stirred evenly to obtain a mixture C;
S4: the mixture B, wild cherry berry juice concentrate, black currant juice concentrate and the mixture C were added to the mixture A, a resulting solution was added with the flavoring agent and stirred to homogenize, then filled and sterilized to obtain the liquid preparation.

TABLE 2
Components and contents in Examples (Ex.) 1-9 of the present
application

| Component | Ex. 1 | Ex. 2 | Ex. 3 | Ex. 4 | Ex. 5 | Ex. 6 | Ex. 7 | Ex. 8 | Ex. 9 |
|---|---|---|---|---|---|---|---|---|---|
| Lutein ester | 0.3 | 0.2 | 0.1 | 1.5 | 0.2 | 0.2 | 0.2 | 0.2 | 0.2 |
| microcapsule | | | | | | | | | |
| powder (kg) | | | | | | | | | |
| Wild cherry | 8 | 2 | 4 | 15 | 2 | 2 | 2 | 2 | 2 |
| berry juice | | | | | | | | | |
| concentrate (kg) | | | | | | | | | |
| Black currant | 2 | 4 | 10 | 15 | 4 | 4 | 4 | 4 | 4 |
| juice | | | | | | | | | |
| concentrate (kg) | | | | | | | | | |
| Sodium | 0.5 | 0.2 | 0.1 | 2 | 0.2 | 0.2 | 0.2 | 0.8 | 0.2 |
| hyaluronate (kg) | | | | | | | | | |
| Vitamin C (kg) | 0.02 | 0.04 | 0.05 | 1 | 0.04 | 0.04 | 0.04 | 0.04 | 0.04 |
| Taurine (kg) | 0.02 | 0.04 | 0.06 | 1.5 | 0.04 | 0.04 | 0.04 | 0.04 | 0.04 |
| Hang white | 5 | 3 | 1 | 10 | 3 | 3 | 3 | 3 | 3 |
| chrysanthemum | | | | | | | | | |
| (kg) | | | | | | | | | |
| pectin (kg) | 0.8 | 0.6 | 0.4 | 3 | — | — | 3 | — | — |
| Xanthan gum | — | — | — | — | 0.6 | — | — | — | — |
| (kg) | | | | | | | | | |
| Erythritol (kg) | 5 | 3 | 2 | 8 | 3 | 3 | 3 | 3 | — |
| Sucralose (kg) | 0.08 | 0.01 | 0.05 | 0.1 | 0.01 | 0.01 | 0.01 | 0.01 | — |
| Citric acid (kg) | 0.02 | 0.05 | 0.08 | 0.3 | 0.05 | 0.05 | 0.05 | 0.05 | — |
| Sodium citrate | 0.05 | 0.08 | 0.1 | 0.5 | 0.08 | 0.08 | 0.08 | 0.08 | — |
| (kg) | | | | | | | | | |
| Raspberry | 0.2 | 0.15 | 0.1 | 0.3 | 0.15 | 0.15 | 0.15 | 0.15 | — |
| essence (kg) | | | | | | | | | |
| Water (kg) | 78 | 87 | 88 | 52 | 87 | 87 | 87 | 87 | — |

### Comparative Example 1

A liquid preparation having effects of protecting eyes and improving eyesight in this comparative example was prepared by using the same steps as well as the same reagents and process parameters as that of Example 2, except that the lutein ester microcapsule powder was replaced by an equal amount of water.

### Comparative Example 2

A liquid preparation having effects of protecting eyes and improving eyesight in this comparative example was prepared by using the same steps as well as the same reagents and process parameters as that of Example 2, except that the wild cherry berry juice concentrate was replaced by an equal amount of water.

### Comparative Example 3

A liquid preparation having effects of protecting eyes and improving eyesight in this comparative example was prepared by using the same steps as well as the same reagents and process parameters as that of Example 2, except that the black currant juice concentrate was replaced by an equal amount of water.

### Comparative Example 4

A liquid preparation having effects of protecting eyes and improving eyesight in this comparative example was prepared by using the same steps as well as the same reagents and process parameters as that of Example 2, except that sodium hyaluronate was replaced by an equal amount of water.

### Comparative Example 5

A liquid preparation having effects of protecting eyes and improving eyesight in this comparative example was prepared by using the same steps as well as the same reagents and process parameters as that of Example 2, except that taurine is replaced by an equal amount of water.

### Comparative Example 6

A liquid preparation having effects of protecting eyes and improving eyesight in this comparative example was prepared by using the same steps as well as the same reagents and process parameters as that of Example 2, except that the Hang white chrysanthemum was replaced by an equal amount of water.

### Comparative Example 7

A liquid preparation having effects of protecting eyes and improving eyesight in this comparative example was prepared by using the same steps as well as the same reagents and process parameters as that of Example 2, except that the wild cherry berry juice concentrate was replaced by an equal amount of blueberry juice concentrate, the Hang white chrysanthemum was replaced by an equal amount of chrysanthemum, and the black currant juice concentrate was replaced by an equal amount of grape juice concentrate.

### Comparative Example 8

A liquid preparation having effects of protecting eyes and improving eyesight in this comparative example was prepared by using the same steps as well as the same reagents and process parameters as that of Example 2, except that the lutein ester microcapsule powder, wild cherry berry juice concentrate, black currant juice concentrate, sodium hyaluronate, vitamin C, taurine and Hang white chrysanthemum were replaced by equal amounts of water.

### Performance Test

1. Samples of Examples and Comparative Examples of the present application were taken as research objects, and an asthenopia relieving effect of each composition was evaluated according to the evaluation method of “relieving asthenopia” in “Technical Specification for Inspection and Evaluation of Health Food of the Ministry of Health”.

### (1) Subject Selection

### Inclusion Criteria:

i) adults aged 18-65 years old with long-term eye use and easy asthenopia (blurred vision, eye distension, headache, orbital distension, heavy eyelids and dry eyes after prolonged vision);
ii) using eyes for more than 6 hours a day;
iii) Willing to follow research protocols; and
iv) all subjects participated in this experiment voluntarily and knowingly.

### Exclusion Criteria:

i) patients with diseases associated with cardiovascular and cerebrovascular, liver and kidney, and blood system, and allergic constitution;
ii) patients with infection, trauma, and diseases associated with cornea, fundus, or the like, and eye surgery within the past 3 months;
iii) pregnant or lactating women; and
iv) those who do not consume products according to regulations or have incomplete data, which affect the efficacy or safety judgment.

### (2) Test Method

A control design was adopted. According to the requirements of randomization and double blind, volunteers who met the inclusion criteria and guaranteed to cooperate with the experiment were divided into the test groups and the control group according to symptoms and visual examination, considering the factors of age and gender, with 10 people in each group. The test groups were given the product at the recommended dose (50 mL/d). Over 60 consecutive days, the test groups were given the samples of Examples 1-9 and Comparative Examples 1-7, while the control group was given the sample of Comparative Example 8 as a placebo, for. During the test, the original diet was not changed and normal diet was maintained.

### (3) Symptom Observation:

Ocular subjective symptoms include eye distension, sore eyes, photophobia, blurred vision, dry eyes, foreign body sensation and tearing; symptoms of general discomfort associated with fatigue include headache, dizziness, irritability, and anxiety.

The volunteers were inquired about the condition of asthenopia, and the symptoms of eye pain, eye distension, photophobia, blurred vision, dry eyes, etc. were observed. Scores were calculated before and after taking the product according to symptoms severity (3 scores for severe symptoms, 2 scores for moderate symptoms, 1 score for mild symptoms) combined with the evaluation method of asthenopia symptoms as shown in Table 3 below. The symptom improvement rate was observed according to the improvement of symptoms (taking any symptom improvement with 1 score or more as improvement). The result was shown in the sole FIGURE.

TABLE 3
Criteria for diagnosis of asthenopia symptoms

| | score | | | |
|---|---|---|---|---|
| Symptom | 0 | 1 score | 2 scores | 3 scores |
| Eye distension | No | Occasionally | Sometimes happen, | Frequently |
| | | happen | get better after rest | happen, get |
| | | | | better after rest |
| Sore eyes | No | Occasionally | Sometimes happen | Frequently |
| | | happen | | happen |
| Photophobia | No | Occasionally | Sometimes happen | Frequently |
| | | happen | | happen |
| Blurred vision | No | Occasionally | Sometimes happen, | Frequently |
| | | happen | get better after rest | happen, get |
| | | | | better after rest |
| Dry eyes | No | Occasionally | Sometimes happen | Frequently |
| | | happen | | happen |
| Foreign body | No | Occasionally | Sometimes happen | Frequently |
| sensation | | happen | | happen |
| Tearing | No | Occasionally | Sometimes happen | Frequently |
| | | happen | | happen |
| General | No | Occasionally | Sometimes happen | Frequently |
| discomfort | | happen | | happen |
| associated with | | | | |
| fatigue | | | | |

Note:
“occasionally happen” means 1-2 times/2 days; “sometimes happen” means 2-3 times/day; and “frequently happen” means >3 times/day.

As can be seen from the sole FIGURE, symptom improvement rates of each example group and comparative example group are higher than that of the control group, and the differences are significant. The symptom improvement rates of Examples 2 and 4 are higher than those of other example groups, and the differences are extremely significant, indicating the best effect in relieving asthenopia. The improvement rates of Examples 2 and 9 are similar, because Examples 2 and 9 have the same active raw material content and the resulting relieving effects on asthenopia are similar. When compared with the control group, Comparative examples 1-7 also have improved symptom improvement rates to a certain extent, indicating that the contained effective ingredients have a certain effect on relieving asthenopia, but the effects of protecting eye are still lower than those of the Examples of the present application.

(4) Duration of photopic vision measurement: duration of photopic vision=photopic vision time/total fixation time, the measurement time was 3 min, and the average value was taken after two measurements, the results were shown in Table 4 below.

TABLE 4
effects of samples of Examples and Comparative examples
of the present application on duration of photopic vision

| | duration of | duration of |
|---|---|---|
| | photopic vision | photopic vision |
| | before taking | after taking |
| Tested item | the product | the product |
| Control group | 0.55 ± 0.04 | 0.56 ± 0.05 |
| Example 1 | 0.54 ± 0.04 | 0.72 ± 0.02 |
| Example 2 | 0.55 ± 0.03 | 0.81 ± 0.07 |
| Example 3 | 0.53 ± 0.05 | 0.71 ± 0.03 |
| Example 4 | 0.51 ± 0.03 | 0.87 ± 0.03 |
| Example 5 | 0.54 ± 0.03 | 0.80 ± 0.05 |
| Example 6 | 0.53 ± 0.02 | 0.81 ± 0.02 |
| Example 7 | 0.52 ± 0.05 | 0.82 ± 0.04 |
| Example 8 | 0.54 ± 0.04 | 0.81 ± 0.02 |
| Example 9 | 0.53 ± 0.02 | 0.79 ± 0.05 |
| Comparative Example 1 | 0.51 ± 0.05 | 0.62 ± 0.05 |
| Comparative Example 2 | 0.53 ± 0.03 | 0.59 ± 0.03 |
| Comparative Example 3 | 0.55 ± 0.05 | 0.60 ± 0.04 |
| Comparative Example 4 | 0.54 ± 0.05 | 0.63 ± 0.06 |
| Comparative Example 5 | 0.52 ± 0.03 | 0.58 ± 0.03 |
| Comparative Example 6 | 0.54 ± 0.04 | 0.64 ± 0.05 |
| Comparative Example 7 | 0.51 ± 0.03 | 0.59 ± 0.05 |

As can be seen from the Table 4, the comparison between the test groups and the control group shows a significant difference in the duration of photopic vision, and the average duration of photopic vision increased by more than or equal to 10% is considered as a good relative effect. The duration of photopic vision in each group of Examples 1-4 is significantly improved, and the difference is significant when compared with the control group, among which Examples 2 and 4 have extremely significant differences, and the average duration of bright vision is increased by 26% and 36% respectively, meaning that they have a significant effect on relieving asthenopia.

2. Stability test: samples of Examples were stored at a temperature of 37° C. and relative humidity of 75% for 3 months, and the contents of anthocyanin and lutein ester in the liquid preparations at the initial stage and after 3 months of storage were determined by HPLC. By comparing the contents of anthocyanin and lutein ester before and after storage, the ratios of the contents after 3 months of storage to the initial contents were the retention rates of anthocyanin and lutein ester under accelerated test conditions. The retention rates of anthocyanin and lutein ester were tested and recorded, as shown in Table 5 below.

TABLE 5
storage retention rates of anthocyanin and lutein
ester in examples of the present application

| | Retention rate | Retention rate of |
|---|---|---|
| Tested item | of anthocyanin (%) | lutein ester (%) |
| Example 1 | 79.15 ± 0.21 | 81.31 ± 0.16 |
| Example 2 | 82.56 ± 0.15 | 83.25 ± 0.27 |
| Example 3 | 80.55 ± 0.26 | 76.18 ± 0.17 |
| Example 4 | 84.28 ± 0.36 | 85.15 ± 0.13 |
| Example 5 | 68.76 ± 0.27 | 69.27 ± 0.26 |
| Example 6 | 63.19 ± 0.24 | 65.36 ± 0.26 |
| Example 7 | 86.56 ± 0.31 | 87.32 ± 0.21 |
| Example 8 | 69.64 ± 0.21 | 72.23 ± 0.36 |
| Example 9 | 64.23 ± 0.16 | 64.39 ± 0.34 |

As shown in Table 5, the results of the retention rates of anthocyanin and lutein ester of the liquid preparations in Examples 2 and 5-6 and 8 after 3 months of storage were compared. The acceleration experiment was conducted at a temperature of 37° C. and relative humidity of 75%, and it was found that pectin had better delayed degradation effect on anthocyanin and lutein ester than xanthan gum or hyaluronic acid thickening agents, and can extend the validity of the product.

The above specific embodiments further elaborate the purpose, technical solution and beneficial effects of the present application, and it should be understood that the above embodiments are only specific embodiments of the present application and are not used to limit the scope of protection of the present application. In particular, for a person skilled in the art, any modification, equivalent replacement, improvement, etc. made within the spirit and principles of the present application shall be included in the scope of protection of the present application.

1. A composition having effects of protecting eyes and improving eyesight, comprising the following components by weight:
lutein ester microcapsule powder: 0.1-1.5 parts;
wild cherry berry juice concentrate: 2-15 parts;
black currant juice concentrate: 2-15 parts;
sodium hyaluronate: 0.1-2 parts;
vitamin C: 0.02-1 part;
taurine: 0.02-1.5 parts; and
Hang white chrysanthemum: 1-10 parts.

2. Use of the composition having effects of protecting eyes and improving eyesight according to claim 1 in the field of preparation of food or medicines.

3. A liquid preparation having effects of protecting eyes and improving eyesight, comprising the following components by weight:
lutein ester microcapsule powder: 0.1-1.5 parts;
wild cherry berry juice concentrate: 2-15 parts;
black currant juice concentrate: 2-15 parts;
sodium hyaluronate: 0.1-2 parts;
vitamin C: 0.02-1 part;
taurine: 0.02-1.5 parts;
Hang white chrysanthemum: 1-10 parts;
a thickening agent: 0.4-3 parts; and
a solvent: 50-90 parts.

4. The liquid preparation having effects of protecting eyes and improving eyesight according to claim 3, wherein the thickening agent is pectin and/or xanthan gum and the solvent is water.

5. The liquid preparation having effects of protecting eyes and improving eyesight according to claim 3, wherein the liquid preparation further comprises a sweetening agent of 0.01 to 9 parts by weight.

6. The liquid preparation having effects of protecting eyes and improving eyesight according to claim 5, wherein the sweetening agent is erythritol and/or sucralose.

7. The liquid preparation having effects of protecting eyes and improving eyesight according to claim 5, wherein the liquid preparation further comprises an acidity regulator of 0.02 to 0.8 parts by weight.

8. The liquid preparation having effects of protecting eyes and improving eyesight according to claim 7, wherein the acidity regulator is citric acid and/or sodium citrate.

9. The liquid preparation having effects of protecting eyes and improving eyesight according to claim 7, wherein the liquid preparation further comprises a flavoring agent of 0.1 to 0.3 parts by weight.

10. A preparation method of the liquid preparation having effects of protecting eyes and improving eyesight according to claim 9, comprising the following steps:
S1: heating one-third of a solvent to 50° C.-70° C., mixing sodium hyaluronate and a sweetening agent evenly and adding to the solvent under stirring, heating a result mixture under stirring until the result mixture is transparent, and conducting cooling to obtain a mixture A;
S2: adding lutein ester microcapsule powder and Hang white chrysanthemum to another one-third of the solvent, and stirring evenly to obtain a mixture B;
S3: adding vitamin C, taurine and the acidity regulator into another one-third of the solvent, and stirring evenly to obtain a mixture C;
S4: adding the mixture B, wild cherry berry juice concentrate, black currant juice concentrate and the mixture C to the mixture A, and adding a flavoring agent and stirring evenly to homogenize to obtain the liquid preparation.
